(19) United States
(12) Patent Application Publication
(10) Pub. No.: US 2012/0315308 A1
(43) Pub. Date: Dec. 13, 2012

(54) METHODS AND DEVICES FOR CONTROLLING PARTICLE SIZE AND PARTICLE SIZE DISTRIBUTION

(76) Inventor: William A. Travers, Baldwin, MD (US)

(21) Appl. No.: 13/580,953

(22) PCT Filed: Feb. 24, 2011
(86) PCT No.: PCT/US2011/026125

§ 371 Date: Aug. 23, 2012

Related U.S. Application Data
(60) Provisional application No. 61/307,582, filed on Feb. 24, 2010.

(51) Int. Cl. B29B 13/08; A61K 9/10

(52) U.S. Cl. 424/400; 264/9

(57) ABSTRACT

Methods and apparatus for continuously preparing a particulate material are provided. One method of the invention includes mixing a first solution comprising a solute dissolved in a solvent, with a second solution, thereby providing a first solution-second solution mixture; and introducing the mixture into the high-energy zone of a high shear dispersion system and dispersing the first solution-second solution mixture prior to significant particulate growth, whereby the dispersed mixture forms a suspension of particles of the solute; and wherein significant particulate growth has occurred when the average equivalent spherical diameter of at least one particle in the solution is greater than 10 micrometer. Integrated apparatuses for performing methods of the invention are also provided.

### CROSS-REFERENCE TO RELATED APPLICATIONS

This application claims priority to U.S. Provisional Application Ser. No. 61/307,582, entitled “Enhanced Dispersion Technique for Controlling the Particle Size Distribution of Crystals Produced by Solvent/Antisolvent Precipitation,” filed Feb. 24, 2010, which is incorporated herein by reference in its entirety.

### BACKGROUND OF THE INVENTION

The formation of particles, for example crystals (e.g., of pharmaceutically active materials), amorphous particles, nanocrystalline materials for use in cosmetics, composite materials, semiconductors, etc. is an important industrial process. For example, crystallization is an important separation and purification process in many industries. Over the past several decades the study of crystallization operations has taken on even higher levels of importance because of several critical factors that require increased control of the crystallization process.

For example, the potential pharmacological value of a large number of compounds has yet to be realized because the compounds cannot be effectively formulated, and therefore, fail to pass initial screening tests (Panagiotuou et al. (2009). Ind. Eng. Chem. Res. 48, pp. 1761-1771). In some instances, in order to confer stability on a compound, the compound is crystallized prior to it being formulated. In order for the formulation to be reproducible, the crystals provided should be similar in size.

Various methods can be used to form crystals. One method is solvent-antisolvent mixing, in which a solute dissolved in a solvent is mixed with an antisolvent to initiate crystallization of the solute. The particle size distributions, morphology and polymorphology of the final product depends upon the properties and stabilities of particles formed by solvent-antisolvent mixing. Accordingly, it is desirable to have a method and apparatus that controls the particle size and particle size distributions of crystals formed by solvent-antisolvent mixing.

The present invention addresses this and other needs.

### SUMMARY OF THE INVENTION

In one embodiment, the present invention is directed to a method of continuously preparing a particulate material (e.g., crystalline or amorphous), an emulsion, liposome, or an encapsulated material. The method comprises mixing a first solution (e.g., a solvent solution comprising a solute dissolved in a solvent), with a second solution (e.g., a miscible non-solvent system), whereby the combination of the first and second solutions forms a particulate material (e.g., by providing a solvent solution-antisolvent mixture which causes the solute to precipitate and/or crystallize); then introducing the mixture into the high-energy zone of a high shear dispersion system and dispersing the mixture of solutions (e.g., solvent solution-antisolvent mixture) prior to significant formation of particles (e.g., significant crystal growth), whereby the dispersed mixture (e.g., solvent solution-antisolvent mixture) forms a suspension of particles (e.g., crystals of the solute); and wherein significant particle formation (e.g., significant crystal growth) has occurred when the average equivalent spherical diameter of the particles is greater than about 10 μm.

In a further embodiment, the mixing step comprises sonicating the first solution (e.g., solvent solution) with a sonicating device, and dispersing the sonicated first solution (e.g., solvent solution) into the second solution (e.g., antisolvent). In even a further embodiment, the mixing step further comprises directly feeding the sonicated mixture (e.g., solvent solution) into the inlet and/or inlet reservoir of the high shear dispersion system, which contains the second solution (e.g., antisolvent).

In another embodiment, the present invention is directed to a method of minimizing the particle size distribution of a crystalline material. The method comprises mixing the first solution (e.g., solvent solution comprising a solute dissolved in a solvent) with a second solution (e.g., miscible antisolvent system), thereby providing a mixture of the first and second solutions (e.g., solvent solution-antisolvent mixture); and introducing the mixture into the high-energy zone of a high shear dispersion system and dispersing the mixture of the first and second solutions (e.g., solvent solution-antisolvent mixture) prior to significant particle (e.g., crystal) growth, whereby the dispersed mixture of the first and second solutions (e.g., solvent solution-antisolvent mixture) forms a suspension of particles (e.g., crystals of the solute); and wherein significant particle (e.g., crystal) growth has occurred when the average equivalent spherical diameter (or average particle size) of the particles is greater than 10 μm.

Also disclosed herein are apparatuses for preparing a particulate (e.g., crystalline) material. In one embodiment, the apparatus includes a mixing device and a high shear dispersion device fluidically coupled to the mixing device. The mixing device is fluidically coupled to a first reservoir containing a first solution (e.g., a solute dissolved in a solvent). The mixing device is configured to convey the first solution (e.g., solvent solution) into a second reservoir and/or inlet feed containing a second solution (e.g., an antisolvent) such that the first and second solutions (e.g., solute-solvent solution and the antisolvent) are mixed. The high shear dispersion device is configured to convey the mixture (e.g., a solution-antisolvent mixture) from the second reservoir and/or inlet feed through a high-energy zone defined by the high shear dispersion device. The high-energy zone is configured to facilitate dispersion of the first and second solution (e.g., solvent solution-antisolvent) mixture.

### BRIEF DESCRIPTION OF THE FIGURES

FIG. 1 is a schematic illustration of an apparatus for preparing a crystalline material according to an embodiment.

FIG. 2 is a schematic illustration of an apparatus for preparing a crystalline material according to an embodiment.

FIG. 3 is a graph showing naproxen particle size distribution vs. frequency of particle size, after carrying out one method of the invention.

FIG. 4 is a graph showing naproxen particle size distribution vs. frequency of particle size, after carrying out one method of the invention.

### DETAILED DESCRIPTION OF THE INVENTION

The present invention provides methods and devices for the continuous or batch preparation of a particulate (e.g., crystalline or amorphous) material, which also minimizes the particle size distribution of the particulate material. The particulate material, in one embodiment, is a crystalline or amorphous material (e.g., an active pharmaceutical ingredient), an inorganic particle (e.g., a metal particle, a salt, etc.). Methods for preparing microemulsions, nanoemulsions, liposomes and encapsulation products are also provided herein.

In one embodiment, the particles formed by the process(es) and apparatus(es) of the present invention comprise crystalline materials formed by combining a solute (e.g., and active pharmaceutical ingredient) dissolved in a solvent with a miscible antisolvent, whereby the resulting mixture causes the solute to precipitate (e.g., as crystalline or amorphous particles), or crystallize. By vigorously mixing the solute/solvent solution with a miscible antisolvent, e.g., by using an ultrasonic horn, particles begin to form. In contrast, conventional processes for forming crystalline particles combine the solute/solvent solution with the antisolvent as a batch process, and simply allow the growing crystals to form without the application of significant mechanical force, e.g., from a high shear dispersion device. The particle size and particle size distribution resulting from such conventional processes are substantially larger and broader, respectively, compared to that provided by the present invention. Furthermore, even if the particle size and/or particle size distribution of crystals formed by the conventional batch processes are modified (e.g., by batch-type post-processing of the formed crystals with a high shear dispersion device), the particle sizes and particle size distributions of the present invention can only be provided, if at all, by numerous passes through the high shear dispersion device. Thus, even if one could obtain the particle size/particle size distribution provided by the present invention, e.g., post-processing the formed crystals with a high shear dispersion device to reduce the particle size, the conventional process is more time-consuming, equipment intensive, and/or expensive than the process of the present invention. Similarly, formation of other types of particles using acid base reactions and redox (reduction-oxidation) reactions, or formation of liposomes, encapsulated materials, or emulsions using the process of the present invention provides much smaller and a more uniform size distribution of particles, liposomes, emulsions, compared to conventional batch methods.

The properties and stabilities of particles formed by solvent-antisolvent crystallizations, acid base reactions and redox reactions are dependent upon the particle size distributions, morphology and polymorphology of the final product. As described further below, various types of ultrasonic devices, Venturi devices, atomizing devices and spray devices (e.g., an air nozzle device such as those available from Ivek Corp. under the tradename Sonicair; such devices can be operated using a variety of fluids such as air, N₂ or even liquids) can be used for a high degree of initial mixing of two liquids, for example delivered via a coaxial feed, just prior to secondary processing in a high shear dispersion device.

For the methods of crystallization provided herein, the respective solvent and antisolvent are miscible. Additionally, the respective acid and base solutions are miscible, as are the solvent solution and a reducing agent solution, if used.

The properties and stabilities of emulsions, micelles, lipid and polymer encapsulations are dependent upon the droplet size distributions. In the case of emulsion, micelle, liposome and polymer encapsulation processes, the liquids are immiscible. The time between the two steps in the processes is minimal to avoid coalescence of the phases as the intent is to mix the solutions on a micro level, thereby creating the phase or encapsulated product. The methods described herein provide smaller particle size distributions when used as a pretreatment to the introduction of a high shear emulsion, liposomal or polymer encapsulation process.

Components for use with the various methods and devices of the invention are provided below.

### Solutes

In one embodiment of the invention, the solute is an active agent. In a further embodiment, the active agent is an active pharmaceutical ingredient. In another embodiment, the solute is an inorganic material, as described further below.

The active ingredients suitable for use in the methods provided herein are not particularly limited, and can be hydrophilic, lipophilic, amphiphilic or hydrophobic. Alternatively, the active ingredient can be provided separately from the solid pharmaceutical composition, such as for co-administration. Classes of active ingredients include, in one embodiment, drugs, nutrients, cosmeceuticals, diagnostic agents, nutritional agents, agricultural products, and the like. One of ordinary skill in the art will appreciate that that the categorization of an active ingredient as hydrophilic or hydrophobic may change, depending upon the particular salts, isomers, analogs and derivatives used.

In one embodiment, the active ingredient agent is hydrophobic. Hydrophobic active ingredients are compounds with little or no water solubility. Intrinsic water solubilities (i.e., water solubility of the unionized form) for hydrophobic active ingredients are less than about 1% by weight, and typically less than about 0.1% or 0.01% by weight.

Suitable hydrophobic active ingredients are not limited by therapeutic category, and can be, for example, analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-Parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nonsteroidal anti-inflammatory drugs, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Specific, non-limiting examples of suitable hydrophobic active ingredients are: acutretin, albendazole, albuterol, aminogluthemide, amiodarone, amlodipine, amphetamine, amphotericin B, atorvastatin, atovaquone, azithromycin, baclofen, beclomethsone, benezepril, benzonatate, betamethasone, bicalutanide, budesonide, bupropion, busulphan, butenafine, calcifediol, calciprotiene, calcitriol, camptothecan, candesartan, capsaicin, carbamezepine, carotenes, celecoxib, cerivistatin, cetrizine, chlorpheniramine, cholecalciferol, cilostazol, cimetidine, cinnarizine, ciprofloxacin, cisapride, clarithromycin, clemastine, clomiphene, clomipramine, clopidrogel, codeine, coenzyme Q10, cyclobenzaprine, cyclosporine, danazol, dantrolene, dexchlopheniramine, diclofenac, dicoumarol, digoxin, dihydro epiandrosterone, dihydroergotamine, dihydrotachysterol, dirithromycin, donepezil, efavirenz, eposartan, ergocalciferol, ergotamine, essential fatty acid sources, etodolac, etoposide, famotidine, fenofibrate, fentanyl, fexofenadine, finasteride, flucanazole, flurbiprofen, fluvastatin, fosphenytion, frovatriptan, furazolidone, gabapentin, gemfibrozil, glibenclamide, glipizide, glyburide, glymepride, griseofulvin, halofantrine, ibuprofen, irbesartan, irinotecan, isosorbide dinitrate isotreinoin, itraconazole, ivermectin, ketoconazole, ketorolac, lamotrigine, lanosprazole, leflunomide, lisinopril, loperamide, loratadine, lovastatin, L-thryroxine, lutein, lycopene, medroxyprogesterone, mefepristone, mefloquine, megesterol acetate, methadone, methoxsalen, metronidazole, metronidazole, miconazole, midazolam, miglitol, minoxidil, mitoxantrone, montelukast, nabumetone, nalbuphine, naproxen, naratiptan, nelfinavir, nifedipine, nilsolidipine, nilutanide, nitrofurantoin, nizatidine, omeprazole, oprevelkin, osteradiol, oxaprozin, paclitaxel, paricalcitol, paroxetine, pentazocine, pioglitazone, pizofetin, pravastatin, prednisolone, probucol, progesterone, pseudo-ephedrine, pyridostigmine, rabeprazole, raloxifene, refocoxib, repaglinide, rifabutine, rifapentine, rimexolone, ritanovir, rizatriptan, rosigiltazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terzosin, tetrahydrocannabinol, tiagabine, ticlidopine, tirofibran, tizanidine, topiramate, topotecan, toremifene, tramadol, tretinoin, troglitazone, trovafloxacin, ubidecarenone, valsartan, venlafaxine, vertoporfin, vigabatrin, vitamin A, vitamin D, vitamin E, vitamin K, zafirlukast, zileuton, zolmitriptan, zolpidem, zopiclone, and mixtures thereof. In one embodiment, a salt, isomer or derivative of one of the above-listed hydrophobic active ingredients is used as a solute of the invention.

In another embodiment, the solute is a hydrophilic active ingredient. Amphiphilic compounds are also included within the class of hydrophilic active ingredients. Apparent water solubilities for hydrophilic active ingredients are greater than about 0.1% by weight, and typically greater than about 1% by weight. In one embodiment, the hydrophilic active ingredient is a hydrophilic drug. In another embodiment, the hydrophilic active ingredient is a cosmeceutical, a diagnostic agent, or a nutritional agent.

Suitable hydrophilic active ingredients are not limited by therapeutic category, and can be, for example, analgesics, anti-inflammatory agents, anthelmintics, anti-arrhythmic agents, anti-bacterial agents, anti-viral agents, anti-coagulants, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, erectile dysfunction improvement agents, immunosuppressants, anti-protozoal agents, anti-thyroid agents, anxiolytic agents, sedatives, hypnotics, neuroleptics, β-blockers, cardiac inotropic agents, corticosteroids, diuretics, anti-Parkinsonian agents, gastro-intestinal agents, histamine receptor antagonists, keratolytics, lipid regulating agents, anti-anginal agents, cox-2 inhibitors, leucotriene inhibitors, macrolides, muscle relaxants, nonsteroidal anti-inflammatory drugs, nutritional agents, opioid analgesics, protease inhibitors, sex hormones, stimulants, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Likewise, the hydrophilic active ingredient can be a cytokine, a peptidomimetic, a peptide, a protein, a toxoid, a serum, an antibody, a vaccine, a nucleoside, a nucleotide, a portion of genetic material, a nucleic acid, or a mixture thereof.

Specific non-limiting examples of suitable hydrophilic active ingredients include: acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotoxime; cefoxitin sodium; ceftizoxime; ceftriaxone; cefuroxime axetil; cephalexin; cephapirin sodium; cholera vaccine; chrionic gonadotropin; cidofovir; cisplatin; cladribine; clidinium bromide; clindamycin and clindamycin derivatives; ciprofloxacin; clondronate; colistimethate sodium; colistin sulfate; cortocotropin; cosyntropin; cromalyn sodium; cytarabine; daltaperin sodium; danaproid; deforoxamine; denileukin diftitox; desmopressin; diatrizoate megluamine and diatrizoate sodium; dicyclomine; didanosine; dirithromycin; dopamine hydrochloride; domase alpha; doxacurium chloride; doxorubicin; editronate disodium; elanaprilat; enkephalin; enoxacin; enoxaprin sodium; ephedrine; epinephrine; epoetin alpha; erythromycin; esmol hydrochloride; factor IX; famiciclovir; fludarabine; fluoxetine; foscarnet sodium; ganciclovir; granulocyte colony stimulating factor; granulocyte-macrophage stimulating factor; growth hormones-recombinant human; growth hormone-bovine; gentamycin; glucagon; glycopyrolate; gonadotropin releasing hormone and synthetic analogs thereof; GnRH; gonadorelin; grepafloxacin; hemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide isofosfamide; japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mephenzolate bromide; mesalmine; methanamine; methotrexate; methscopolamine; metformin hydrochloride; metroprolol; mezocillin sodium; mivacurium chloride; mumps viral vaccine; nedocromil sodium; neostigmine bromide; neostigmine methyl sulfate; neutontin; norfloxacin; octreotide acetate; ofloxacin; olpadronate; oxytocin; pamidronate disodium; pancuronium bromide; paroxetine; pefloxacin; pentamindine isethionate; pentostatin; pentoxifylline; periciclovir; pentagastrin; phentolamine mesylate; phenylalanine; physostigmine salicylate; plague vaccine; piperacillin sodium; platelet derived growth factor-human; pneumococcal vaccine polyvalent; poliovirus vaccine inactivated; poliovirus vaccine live (OPV); polymixin B sulfate; pralidoxine chloride; pramlintide; pregabalin; propofenone; propenthaline bromide; pyridostigmine bromide; rabies vaccine; residronate; ribavarin; rimantadine hydrochloride; rotavirus vaccine; salmetrol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valaciclovir; valsartan; varicella virus vaccine live; vasopressin and vasopressin derivatives; vecoronium bromide; vinblastin; vincristine; vinorelbine; vitamin B12; warfarin sodium; yellow fever vaccine; zalcitabine; zanamavir; zolandronate; zidovudine; pharmaceutically acceptable salts, isomers and derivatives thereof; and mixtures thereof.

In other embodiments, the solute is an inorganic material. For example, catalysts, semiconductor materials, materials for use in electronics, paints, pigments, coatings, polishing agents for silicon wafers can be used as a solute.

Non-limiting examples of inorganic precursors include palladium chloride, hydrogen hexachloroplatinate IV, Potassium tetrachloroplatinate II, silver nitrate, silver tetraoxylchlorate, clororauric acid and rhodium chloride.

Typical reducing agents for these applications include, but are not limited to, hydrogen, sodium citrate, hydroxylamine hydrochloride, citric acid, carbon monoxide, phosphorous in ether, methanol, hydrogen peroxide, sodium carbonate, sodium carbonate, formaldehyde, sodium tetrahydroborate and ammonium ions. In the case of inorganic particles, for example tin, a first solution of Sn (tin) ions is provided. The solute (tin ions), in one embodiment is provided as a salt (tin salt) dissolved in a solvent to provide a solvent solution. The tin salt, in one embodiment, is SnCl₂ or SnCl₄. Solvents are discussed further below.

### Solvents

As described herein, in one embodiment, a solute is dissolved in a solvent, to form a “solvent solution.”

Appropriate selection of a solvent for use in the methods provided herein may enhance the yield or determine characteristics such as crystal form, purity and solubility. The present invention is not limited by a particular solvent type. A solvent, in one embodiment, is selected depending on the solute to be crystallized. In one embodiment, the solvent is selected from water, various aqueous, acidic or basic solutions, or one of the solvents provided in Table 1, below, or a combination of thereof. That is, the solvent can comprise a single compound, or a mixture of two or more compounds. In one embodiment, the solvent is DMSO.

If an active ingredient is being crystallized, residual solvent levels must be suitable for the active ingredient to eventually be safely administered to a subject in need of the active ingredient. In general, class 1 solvents are generally avoided unless their use can be strongly justified in a risk-benefit assessment.

Class 1 solvents are either known human carcinogens, suspected human carcinogens, and/or environmental hazards. Some class 2 solvents are suspected of significant, but reversible toxicities. Class 2 solvents may also be implicated in neurotoxicity or teratogenicity. Class 3 solvents have low toxic potential to humans, and class 3 residual solvents have permitted daily exposures (PDEs) on the order of 50 mg or more per day.

In a preferred embodiment, a class 2 or class 3 solvent provided in Table 1 is utilized in the methods of the invention.

TABLE 1
Solvents for use with the present invention.

| Solvent | Other Names | Structure | Class |
|---|---|---|---|
| Acetic acid | Ethanoic acid | CH₃COOH | Class 3 |
| Acetone | 2-Propanone | CH₃COCH₃ | Class 3 |
| Acetonitrile | Propan-2-one | CH₃CN | Class 2 |
| Anisole | Methoxybenzene | OCH₃ | Class 3 |
| Benzene | Benzol | C₆H₆ | Class 1 |
| 1-Butanol | n-Butyl alcohol, Butan-1-ol | CH₃(CH₂)₃OH | Class 3 |
| 2-Butanol | sec-Butyl alcohol, Butan-2-ol | CH₃CH₂CH(OH)CH₃ | Class 3 |
| Butyl acetate | Acetic acid butyl ester | CH₃COO(CH₂)₃CH₃ | Class 3 |
| tert-Butylmethyl ether | 2-Methoxy-2-methylpropane | (CH₃)₃COCH₃ | Class 3 |
| Carbon tetrachloride | Tetrachloromethane | CCl₄ | Class 1 |
| Chlorobenzene | | C₆H₅Cl | Class 2 |
| Chloroform | Trichloromethane | CHCl₃ | Class 2 |
| Cumene | Isopropylbenzene, (1- | C₉H₁₂ | Class 3 |
| | Methylethyl)benzene | | |
| Cyclohexane | Hexamethylene | C₆H₁₂ | Class 2 |
| 1,2-Dichloroethane | sym-Dichloroethane, Ethylene dichloride, | CH₂ClCH₂Cl | Class 1 |
| | Ethylene chloride | | |
| 1,1-Dichloroethene | 1,1-Dichloroethylene, Vinylidene chloride | H₂C═CCl₂ | Class 1 |
| 1,2-Dichloroethene | 1,2-Dichloroethylene, Acetylene | ClHC═CHCl | Class 2 |
| | dichloride | | |
| 1,2-Dimethoxyethane | Ethyleneglycol dimethyl ether, | H₃COCH₂CH₂OCH₃ | Class 2 |
| | Monoglyme, Dimethyl cellosolve | | |
| N,N-Dimethylacetamide | DMA | CH₃CON(CH₃)₂ | Class 2 |
| N,N-Dimethylformamide | DMF | HCON(CH₃)₂ | Class 2 |
| Dimethyl sulfoxide | Methylsulfinylmethane, Methyl sulfoxide, | (CH₃)₂SO | Class 3 |
| | DMSO | | |
| 1,4-Dioxane | p-Dioxane, [1,4]Dioxane | C₄H₈O₂ | Class 2 |
| Ethanol | Ethyl alcohol | CH₃CH₂OH | Class 3 |
| 2-Ethoxyethanol | Cellosolve | CH₃CH₂OCH₂CH₂OH | Class 2 |
| Ethyl acetate | Acetic acid ethyl ester | CH₃COOCH₂CH₃ | Class 3 |
| Ethylene glycol | 1,2-Dihydroxyethane, 1,2-Ethanediol | HOCH₂CH₂OH | Class 2 |
| Ethyl ether | Diethyl ether, Ethoxyethane, 1,1′- | CH₃CH₂OCH₂CH₃ | Class 3 |
| | Oxybisethane | | |
| Ethyl formate | Formic acid ethyl ester | HCOOCH₂CH₃ | Class 3 |
| Formamide | Methanamide | HCONH₂ | Class 2 |
| Formic acid | | HCOOH | Class 3 |
| Heptane | n-Heptane | CH₃(CH₂)₅CH₃ | Class 3 |
| Hexane | n-Hexane | CH₃(CH₂)₄CH₃ | Class 2 |
| Isobutyl acetate | Acetic acid isobutyl ester | CH₃COOCH₂CH(CH₃)₂ | Class 3 |
| Isopropyl acetate | Acetic acid isopropyl ester | CH₃COOCH(CH₃)₂ | Class 3 |
| Methanol | Methyl alcohol | CH₃OH | Class 2 |
| 2-Methoxyethanol | Methyl cellosolve | CH₃OCH₂CH₂OH | Class 2 |
| Methyl acetate | Acetic acid methyl ester | CH₃COOCH₃ | Class 3 |
| 3-Methyl-1-butanol | Isoamyl alcohol, Isopentyl alcohol, 3- | (CH₃)₂CHCH₂CH₂OH | Class 3 |
| | Methylbutan-1-ol | | |
| Methylbutylketone | 2-Hexanone | CH₃(CH₂)₃COCH₃ | Class 2 |
| Methylcyclohexane | Hexan-2-one | C₇H₁₄ | Class 2 |
| Methylene chloride | Cyclohexylmethane | CH₂Cl₂ | Class 2 |
| Methylethylketone | Dichloromethane | CH₃CH₂COCH₃ | Class 3 |
| Methyl isobutyl ketone | 2-Butanone, MEK, Butan-2-one | CH₃COCH₂CH(CH₃)₂ | Class 3 |
| 2-Methyl-1-propanol | 4-Methylpentan-2-one, 4-Methyl-2- | (CH₃)₂CHCH₂OH | Class 3 |
| | pentanone, MIBK | | |
| N-Methylpyrrolidone | Isobutyl alcohol, 2-Methylpropan-1-ol | | Class 2 |
| Nitromethane | 1-Methylpyrrolidin-2-one, 1-Methyl-2- | CH₃NO₂ | Class 2 |
| | pyrrolidinone | | |
| Pentane | n-Pentane | CH₃(CH₂)₃CH₃ | Class 3 |
| 1-Pentanol | Amyl alcohol, Pentan-1-ol, Pentyl alcohol | CH₃(CH₂)₃CH₂OH | Class 3 |
| 1-Propanol | Propan-1-ol, Propyl alcohol | CH₃CH₂CH₂OH | Class 3 |
| 2-Propanol | Propan-2-ol, Isopropyl alcohol | (CH₃)₂CHOH | Class 3 |
| Propyl acetate | Acetic acid propyl ester | CH₃COOCH₂CH₂CH₃ | Class 3 |
| Pyridine | Tetrahydrothiophene 1,1-dioxide | C₅H₉NO | Class 2 |
| Sulfolane | Tetramethylene oxide | C₄H₈O₂S | Class 2 |
| Tetrahydrofuran | Oxacyclopentane | C₄H₈O | Class 2 |
| Tetralin | 1,2,3,4-Tetrahydronaphthalene | C₁₀H₁₂ | Class 2 |
| Toluene | Methylbenzene | C₇H₈ | Class 2 |
| 1,1,1-Trichloroethane | Methylchloroform | CH₃CCl₃ | Class 1 |
| Trichloroethylene | 1,1,2-Trichloroethene | HClC═CCl₂ | Class 2 |
| Xylene* | Dimethylbenzene | C₈H₁₀ | Class 2 |
| | Xylol | | |

If a class 1 solvent is used, its levels should not exceed the levels provided in Table 2, below. Table 3 provides class 2 solvents and their respective PDEs.

TABLE 2
Various Class 1 Solvents

| | Concentration | |
|---|---|---|
| Solvent | Limit (ppm) | Concern |
| Benzene | 2 | Carcinogen |
| Carbon tetrachloride | 4 | Toxic and environmental hazard |
| 1,3-Dichloroethane | 5 | Toxic |
| 1,1-Dichloroethane | 8 | Toxic |
| 1,1,1- | 1500 | Environmental hazard |
| Trichloroethane | | |

TABLE 3
Permitted Daily Exposure of Class 2 Solvents as Provided by
U.S. Pharmacopeia

| | Permitted Daily | |
|---|---|---|
| | Exposure | Concentration |
| Solvent | (mg/day) | Limit (ppm) |
| Acetonitrile | 4.1 | 410 |
| chlorobenzene | 3.6 | 360 |
| chloroform | 0.6 | 60 |
| chclohexane | 38.8 | 3880 |
| 1,2-dichloroethane | 18.7 | 1870 |
| 1,2-dimethoxyethane | 1.0 | 100 |
| N,N- | 10.9 | 1090 |
| dimethylacetamide | | |
| N,N- | 8.8 | 880 |
| dimethylformamide | | |
| 1,4-dioxane | 3.8 | 380 |
| 2-ethoxyethanol | 1.6 | 160 |
| ethylene glycol | 6.2 | 620 |
| formamide | 2.2 | 220 |
| hexane | 2.9 | 290 |
| methanol | 30.0 | 3000 |
| 2-methoxyethanol | 0.5 | 50 |
| methylbutylketone | 0.5 | 50 |
| methylcyclohexane | 11.8 | 1180 |
| methylene chloride | 6.0 | 600 |
| N-methylpyrrolidone | 5.3 | 530 |
| nitromethane | 0.5 | 50 |
| pyridine | 2.0 | 200 |
| sulfolane | 1.6 | 160 |
| tetrahydrofuran | 7.2 | 720 |
| tetralin | 1.0 | 100 |
| toluene | 8.9 | 890 |
| trichloroethylene | 0.8 | 80 |
| xylene | 21.7 | 2170 |

If residual solvent concentration limits in ppm do not exceed the values provided in table 3, the solvent is safe for consumption. The USP General Chapter <467> provides a method for the identification and quantitation of Class 1 and Class 2 residual solvents utilizing headspace sampling and gas chromatography with flame ionization detection (FID). Some Class 2 solvents, including formamide, 2-ethoxyethanol, 2-methoxyethanol, ethylene glycol, N-methylpyrrolidone and sulfolane, are not amenable to detection using this method and require the development of alternative methods. For Class 3 residual solvents levels may be determined as directed under USP <731> Loss on Drying if so indicated in the monograph for the test article. If not indicated or if levels greater than 50 mg per day are expected, the analysis procedure indicated for Class 1 and Class 2 solvents with the appropriate changes made in standards or other validated procedures can be used. Finally, class 3 solvents are regarded as less toxic and of lower risk to human health, as compared to class 1 and class 2 solvents. The USP considers that amounts of class 3 solvents of 50 mg/day or less would be acceptable without further justification.

TABLE 4
Class 3 Solvents

| Solvent | Solvent | Solvent | Solvent |
|---|---|---|---|
| Acetic Acid | Heptane | 1-Butanol | Methyl acetate |
| Acetone | Isobutyl acetate | 2-Butanol | 3-Methyl-1-butanol |
| Anisole | Isoproptyl acetate | Butyl acetate | Methylethylketone |
| Tert- | Methylisobutylketone | Ethyl acetate | 1-Propanol |
| Butylmethyl | | | |
| ether | | | |
| Cumene | 2-Methyl-1-propanol | Ethyl ether | 2-Propanol |
| Dimethyl | Pentane | Ethyl | Propyl acetate |
| sulfoxide | | formate | |
| Ethanol | 1-Pentanol | Formic acid | |

Once an appropriate solute-solvent system is chosen, the solute (e.g., active pharmaceutical ingredient) is dissolved in the solvent, or mixtures of solvents (i.e., cosolvents) to form a solvent solution, and the solvent solution is further mixed with a miscible antisolvent and subjected to high-shear dispersion. The miscible antisolvent is selected based on the solute, i.e., once a solute is selected, a miscible antisolvent is selected in which the solute is insoluble, and which is miscible with the selected solvent.

### Solvent-Antisolvent Substitutions

Although the methods provided herein are mainly described with the use of solvents and antisolvents, the invention is not limited thereto. For example, as described above, if an inorganic material is crystallized or precipitated in a solution, the precursor to the inorganic material (e.g., tin ions, e.g., in the form of a salt) is dissolved in a solvent or solvent mixture to form a solvent solution (e.g., a first solution). The solvent solution is then mixed with a solution comprising a reducing agent (second solution or “reducing agent solution”), for example by using an ultrasonic horn or other mixing device as described herein, followed by high shear dispersion of the mixture. The reducing agent solution, in one embodiment, comprises an alkali metal and naphthalene. In this embodiment, the alkali metal, e.g., sodium, acts as the reducing agent for the tin. Therefore, in inorganic particle embodiments, a reducing agent solution is substituted for an antisolvent. The methods provided herein provide a narrow particle size distribution of inorganic particles when the inorganic precursor is reduced.

In one embodiment, a precipitate is formed by the methods and apparatuses of the invention, for example by using acid and base solutions. In this embodiment, the solute is dissolved in an acid or a base to form a solvent solution (i.e., the acid or base is the solvent). The solvent solution can then be mixed by the methods described herein with a second solution, i.e., a base solution (in the case of an acid as a solvent) or an acid solution (in the case of a base as the solvent) to form a salt of the solute. The methods provided herein provide a narrow particle size distribution when acid base mixing is performed.

In another embodiment, the solute itself is a weak acid or base (for example a weakly basic drug). The solute is then dissolved in an aqueous solution having a pH at which the solute is soluble (e.g., the first solution), then mixed (e.g., using an ultrasonic horn or other device or method as disclosed herein) with another aqueous solution having a pH at which the solute is insoluble. Upon mixing, the change in pH causes the solute to begin to crystallize or precipitate. Prior to formation of particles (e.g., crystals) having an average particle size or average equivalent spherical diameter of greater than about 10 μm, the mixture is introduced into the high energy zone of a high shear dispersion device, thereby providing a relatively uniform and narrow particle size distribution, as described herein.

Other processes known in the art which form particles by the combination of two solutions can be used in the process(es) and apparatus(es) of the present invention, by premixing the two solutions as described herein, the introducing the mixture into the high energy zone of a high shear dispersion device prior to formation of particles (e.g., crystals) having an average particle size or average equivalent spherical diameter of greater than about 10 μm.

In one embodiment, deionized water, or triton X-100, e.g., 0.5% triton-X 100, in deionized water is the antisolvent. In a further embodiment, the solute is naproxen, and is dissolved in DMSO. The solvents provided above in Tables 1-4 can also be anti-solvents depending on the solute being dissolved. That is, a compound or mixture of compounds that is a solvent for one selected solute may be an antisolvent for another solute. Likewise, a compound or mixture of compounds that is an antisolvent for one selected solute may be a solvent for another solute. Selection of the appropriate solvent or mixture of solvents, and antisolvent or mixture of antisolvents for a particular solute can be selected by a variety of methods (e.g., measuring solubility by methods known in the art; selection based on solubility parameters, etc.).

Besides organic, water miscible solvents, another embodiment is the use of pH extremes to solubilize an active pharmaceutical ingredient (solute) with an ionizable functional group. An example is a solute with a carboxylic acid group, in which raising the pH of an aqueous solution (the first solution) above pKa of the acid serves to solubilize it. Another aqueous solution at a lower pH is the antisolvent (the second solution), and when mixed with the solubilized carboxylate salt, the antisolvent produces a pH lower than the pKa of the acid and causes the precipitation of the carboxylic acid.

### Initial Mixing Step

In the methods provided herein, in one embodiment, a first solution is mixed with a second solution, e.g., a solvent solution is initially mixed with an antisolvent. In another embodiment, the solvent solution (first solution) is mixed with a reducing agent solution (second solution). Acid-base mixtures are also amenable for use with the methods of the invention, as provided above. The mixing, in one embodiment, comprises atomization, i.e., dispersing the solvent solution into an antisolvent as a fine stream or particles. Encapsulation products and micelles can also be formed by introducing an atomized stream of one solution (first solution) into an immiscible solution (second solution).

Although various solutions can be mixed by the methods and apparatuses of the invention, for the purposes of brevity, the description is mainly limited to solvent-antisolvent mixing. However, as described above, various substitutions are possible, and within the scope of the invention and the ordinary skill in the art.

In one embodiment, mixing of the first solution (solvent solution) with an appropriate second solution (antisolvent) comprises sonication of the first solution, followed by introduction of the first solution into a second solution, i.e., an antisolvent. The sonicated first solution (e.g., sonicated solvent solution), in one embodiment, is introduced into the second solution (e.g., antisolvent) by atomization, e.g., an air pressure atomizer, electrohydrodynamic atomizer, or some other process by which a steady stream of fine, (e.g., sonicated) solvent particles can be introduced into the antisolvent. In one embodiment, sonication and atomization are integrated as one process, e.g., by using a sonicator with a flow through channel (described further below).

Atomizing the solvent solution provides a fine dispersion of the solvent solution. In this manner, a higher percentage of antisolvent is able to interact with the solvent solution, due to the high surface to volume ratio of the solvent solution droplets, surrounded by the antisolvent. Without wishing to be bound by theory, the high surface to volume ratio promotes crystal nucleation.

Sonication uses sound energy, e.g., ultrasound energy, to speed dissolution in a mixture, and also to disperse and to deagglomerate particles in solution. Sonicators for use with the present invention include the devices described in U.S. Pat. Nos. 5,516,043 and 5,371,429, the disclosures of which are incorporated by reference herein, in their entireties. While these ultrasonic devices offer advanatges over other less sophisticated ultrasonic devices, other ultrasonic devices may also be employed in the methods of the invention. Additional dispersion or spraying devices amenable for use with the present invention include, but are not limited to, the Sonicair Nozzle (IVEK Corp.), the Multisonic System (IVEK Corp.), inline mixers such as rotor-stator and blade mixers, or an ultrasonic probe tip, for example, the ultrasonic probe available from Microson. In one embodiment, an ultrasonic transducer horn is used to pass ultrasound into the solvent solution.

The Sonicair nozzle consists of a single part. Air, liquid and heater ports are located on the top of the nozzle, and the discharge tip is located on the bottom of the nozzle. The Sonicair nozzle is made of stainless steel and is available in a variety of bore sizes. Sonicair nozzles provide a precise discharge pattern for atomizing liquids. The amount and rate of discharge depends on the dispensing system and parameters being used (e.g., the pump, flow speed, tubing diameter, etc.).

The particle size for the Sonicair typically ranges from 5-50 microns, and can be adjusted according to parameters such as temperature, viscosity and surface tension of the liquid. Additionally, there are a number or factors that can help change the spray pattern profile, among these are temperature, dispense rate and air flow. The solute solution, once passed through the ultrasonicator, e.g., a Sonicair nozzle, is introduced into an antisolvent solution, and further mixed.

As stated above, in one embodiment, the Multisonic system from IVEK is employed in the initial mixing step. This system includes (1) a profile board comprising the circuitry necessary to generate the trigger and amplitude profile to the Ultrasonic Driver channels, (2) a driver channel board to provide power to the ultrasonic transducer, (3) ultrasonic transducer with a horn, to convert the electrical energy from the Ultrasonic driver to mechanical vibration.

In one embodiment, the ultrasonication device has a center coaxial channel, so that the solvent solution is fed through the ultrasonicator, either by gravity, or by a metering device, e.g., a pump. This setup allows for a solvent solution to be effectively metered through an ultrasonication device, e.g., by effectively controlling flow rate, and allow allows for direct integration with a second mixing step, as described in further detail below. Metering also allows for control of the particle size of the dispersion introduced into the antisolvent solution.

In one embodiment, the first solution (e.g., solvent solution) is sonicated which leads to atomization of the solution. The solvent solution, in one embodiment, is transferred as an atomized stream to the inlet reservoir or inlet feed of a high-shear dispersion system, described in further detail below. In a further embodiment, the antisolvent is present in the inlet reservoir before the transfer of the solvent solution.

If ultrasonication is used, the frequency of the ultrasound is typically in the range of about 10 kHz to about 300 kHz. In a further embodiment, the frequency range is from about 15 kHz to about 200 kHz, or from about 15 kHz to about 150 kHz, or from about 15 kHz to about 100 kHz, or from about 15 kHz to about 90 kHz, or from about 15 kHz to about 80 kHz, or from about 15 kHz to about 70 kHz, or from about 15 kHz to about 60 kHz, or from about 15 kHz to about 50 kHz, or from about 15 kHz to about 40 kHz, or from about 15 kHz to about 30 kHz, or from about 15 kHz to about 20 kHz.

In another embodiment, ultrasonication is employed, and the frequency of the ultrasound is at least 10 kHz, at least 15 kHz, at least 20 kHz, at least 25 kHz, at least 30 kHz, at least 35 kHz, at least about 40 kHz, at least about 45 kHz, at least about 50 kHz, at least about 55 kHz, at least about 60 kHz, at least about 65 kHz, at least about 70 kHz, at least about 75 kHz, at least about 80 kHz, at least about 85 kHz, at least about 90 kHz, at least about 95 kHz, at least about 100 kHz, at least about 110 kHz, at least about 120 kHz, at least about 130 kHz, at least about 140 kHz, at least about 150 kHz, at least about 160 kHz, at least about 170 kHz, at least about 180 kHz, at least about 190 kHz, at least about 200 kHz, at least about 225 kHz, at least about 250 kHz, at least about 275 kHz, or at least about 300 kHz.

Ultrasonication can be operated at different power, and in one embodiment, the power range employed in the sonication method is from about 0.5 W to about 100 Kw, or from about 0.5 W to about 100 Kw, or from about 1 W to about 50 Kw, or from about 1 W to about 25 Kw, or from about 5 W to about 16 Kw, or from about 10 W to about 15 Kw. In another embodiment, the power used for ultrasonication is at least about 0.5 W, at least about 1 W, at least about 5 W, at least about 10 W, at least about 20 W, at least about 30 W, at least about 40 W, at least about 50 W, at least about 60 W, at least about 70 W, at least about 80 W, at least about 90 W, at least about 100 W, at least about 500 W, at least about 1 kW, at least about 1 kW, at least about 2 kW, at least about 5 kW, at least about 10 kW, at least about 15 kW, at least about 16 kW, at least about 20 kW, at least about 25 kW, at least about 50 kW, at least about 75 kW, or at least about 100 kW.

In another embodiment, ultrasonication is not used as part of the initial mixing step. In a further embodiment, the first solution (e.g., solvent solution) is initially mixed with a second solution (e.g., an antisolvent) by introducing the first solution into the second solution using an in-line mixer, a Venturi device, an atomizer, or a spray device using air or gases to disperse the liquid. For example, in one embodiment, an inline mixer is employed. In one embodiment, the inline mixer is a mixer from IKA®, or a modified version thereof (catalog available at www.ikausa.com/pdfs/2003_Process_Catlog2.pdf). The inline mixer, in one embodiment, can be high shear or medium shear. The outlet of the inline mixer can be fed into the inlet reservoir or inlet feed of the high-shear dispersion device, described in further detail below, to allow for a fully integrated process. The inlet reservoir of the high-shear dispersion device, in one embodiment, contains the antisolvent, so that the solvent solution and antisolvent are mixed when the solvent solution is introduced into the inlet reservoir.

In one embodiment, a Venturi device, i.e., a device that takes advantage of the Venturi effect, is employed for initial mixing of the solvent solution. The solvent solution is then introduced, e.g., by spray, pipette, gravity, into an antisolvent solution.

Other mixing techniques are amenable for use with the invention. The technique should optimize the interaction of the antisolvent with the solvent solution (or the solvent solution with a reducing agent solution, or an acid with a base, etc.), e.g., by creating fine particles of the solvent solution, followed by dispersion of the particles into an appropriate antisolvent.

In one embodiment, the ratios of solvent solution to antisolvent are metered in an appropriate ratio to maintain the required ratio of the different liquids. For a continuous process, the solvent solution to antisolvent ratio can range, for example, from about 1:1 to about 1:10, from about 1:1 to about 1:100, or about 1:1 to about 1:500, or about 1:1 to about 1:1000. An appropriate ratio can be maintained, e.g., by optimizing the flow rate of the solvent solution into the antisolvent, or by controlling the path length between the solvent solution stream and the inlet reservoir of the high shear dispersion device. Alternatively, the relative percentages of solvent to antisolvent, excluding solute, can be about 1:99, about 10:90, about 20:80, about 30:70, about 40:60, about 50:50, about 60:40, about 70:30, about 80:20, about 90:10, or about 99:1.

As described above, the initial mixing step of the method provided herein allows for very small solvent solution droplets to interact with an antisolvent. Without wishing to be bound by theory, the high surface to volume ratio of solvent solution particles to antisolvent favors nucleation of crystals, rather than growth.

### Time Between Mixing Steps

As described herein, methods and apparatuses are provided for batch or continuously preparing a particulate (e.g., crystalline or amorphous) material, and for controlling the particle size and particle size distribution of a particulate material, for example, an active pharmaceutical ingredient, inorganic particle, nanoparticle, microemulsion, nanoemulsion, liposome or encapsulation product.

In one embodiment, a trigger is provided to deliver the mixed solvent solution product in a synchronized manor, to the high shear dispersion system. This may be desirable if the feed to the high shear processor is intermittent with significant delays between flow, and will therefore minimize the amount of time before the first solution-second solution (e.g., solvent-antisolvent) enters the high shear zone. The first solution and second solution (e.g., solvent solution and antisolvent), in one embodiment, are delivered to the high speed dispersion system immediately prior to introduction into the high shear zone. In another embodiment, the first solution (e.g., solvent solution) is delivered to the high speed dispersion system immediately prior to introduction into the system. Therefore, the present invention minimizes lag-time between the mixing steps.

The time between the initial mixing and introduction into the high shear zone of a high shear dispersion module should be minimal, and prior to any significant crystal growth. The methods provided herein allow for mixture of the solvent solution and antisolvent at the micro level, thereby inducing crystal nucleation or acid-base precipitation. The solvent solution may contain seed material or the initial mixing may be used to shorten induction time, facilitating the initiation of primary nucleation at the point of initial mixing. This initial mixing prior to a secondary high shear dispersion process provides smaller particle sizes and/or narrower particle size distributions when used prior to a solvent-antisolvent or acid-base high shear crystallization process.

In one embodiment, the time between first solution-second solution (e.g., solvent solution-antisolvent) mixing, and high shear dispersion of the first solution-second solution combined solution (e.g., solvent solution-antisolvent combined solution), is such that no significant crystal or particulate growth has occurred. In one embodiment, significant crystal or particulate growth has occurred when the average equivalent spherical diameter of a particle in the solution is greater than 10 μm.

Accordingly, in one embodiment, the first solution-second solution combined solution (e.g., solvent solution-antisolvent combined solution) enters the high shear zone of the high shear dispersion module when the average equivalent spherical diameter of a particle in the solution is 10 μm or less in diameter. In another embodiment, high shear dispersion of the solution commences when the average equivalent spherical diameter of a particle is about 9 μm or less in diameter, about 8 μm or less in diameter, about 7 μm or less in diameter, about 6 μm or less in diameter, about 5 μm or less in diameter, about 4 μm or less in diameter, about 3 μm or less in diameter, 2 μm or less in diameter, about 1 μm or less in diameter, about 900 nm or less in diameter, about 800 nm or less in diameter, about 700 nm or less in diameter, about 600 nm or less in diameter, about 500 nm or less in diameter, about 400 nm or less in diameter, about 300 nm or less in diameter, about 200 nm or less in diameter or about 100 nm or less in diameter.

Typically, because the method steps of the invention are fully integrated, and because mixing of the solvent solution and antisolvent occurs in a thorough manner, it will not be necessary to tune the two mixing steps and process flow. However, in some instances, it may be desirable to tune the system so that crystals of a certain size, for example, the sizes given above, do not form.

In one embodiment, in order to determine how much time is necessary for a crystal or particle of a particular size to form, e.g., a crystal or particle with a diameter of 10 μm, a static light scattering experiment (Mie Theory, for example, laser diffraction) is carried out on the solvent solution-anti solvent solution mixture. In another embodiment, dynamic light scattering is employed.

In laser diffraction, particle size distributions are calculated by comparing a sample's scattering pattern with an appropriate optical model using a mathematical inversion process. Mie Theory provides a rigorous solution for the calculation of particle size distributions from light scattering data and is based on Maxwell's electromagnetic field equations. It predicts scattering intensities for all particles, small or large, transparent or opaque within the following assumptions: (1) the particles being measured are spherical, (2) the suspension is dilute, such at the scattered light is measured before it is re-scattered by other particles, (3) the optical properties of the particles and the medium surrounding them is known and (4) the particles are homogeneous.

In dynamic light scattering, the time dependence of the light scattered from a very small region of solution is measured over a period of time, e.g., microseconds to milliseconds to seconds. The fluctuations in the intensity of scattered light are related to the rate of diffusion of particles in and out of the region being studied, and diffusion coefficients of the particles (i.e., solute crystals) can be derived. The diffusion coefficients can be used to extrapolate the solute crystal's hydrodynamic diameter or Stokes radius (“hydrodymanic radius,” R_{H}).

Once the length of time to significant crystal growth is determined, the user of the invention can specifically tailor the apparatus to perform the second method step before significant growth, e.g., by adjusting flow rate, path length between the two process steps, pump speed, etc. In one embodiment, the path length between the two process steps is as minimal as the system allows.

In another embodiment, the time between initial mixing and high shear dispersion is about 15 seconds, about 14 seconds, about 13 seconds, about 12 seconds, about 11 seconds, about 10 seconds, about 9 seconds, about 8 seconds, about 7 seconds, about 6 seconds, about 5 seconds, about 4 seconds, about 3 seconds, about 2 seconds, about 1 seconds, about 0.5 seconds, about 0.1 seconds, about 0.01 seconds, or about 0.001 seconds.

### High-Shear Dispersion Systems (Second Step)

As provided above, the present invention is directed to, in part, continuous or batch methods for preparing a crystalline or particulate material and methods for minimizing the particle size distribution of a crystal or particle population. By continuous, we mean that the first and second solutions are mixed in the first step, then introduced to the high energy zone of the high shear dispersion system second step (optionally including recycling a portion of the resulting product back to the second step). By batch, we mean that after mixing the first and second solutions and introducing the resulting mixture into the high energy zone of the high shear dispersion system (second step), the entire resulting product is recycled one or more times back through the high energy zone of the high shear dispersion system. In one embodiment, the method comprises mixing a first solution (e.g., solvent solution comprising a solute dissolved in a solvent), with a miscible second solution (e.g., antisolvent system), thereby providing a solvent solution-antisolvent mixture; and introducing the mixture into the high-energy zone of a high shear dispersion system and dispersing the first solution-second solution mixture (e.g., solvent solution-antisolvent mixture) prior to significant crystal or particle growth, whereby the dispersed s first solution-second solution mixture (e.g., solvent solution-antisolvent mixture) forms a suspension of crystals or particles of the solute; and wherein significant crystal or particle growth has occurred when the average equivalent spherical diameter of at least one crystal or particle in the solution is greater than 10 μm, or greater than 9 μm, or greater than 8 μm, or greater than 7 μm, or greater than 6 μm, or greater than 5 μm, or greater than 4 μm, or greater than 3 μm, or greater than 2 μm, or greater than 1 μm, or greater than 900 nm, or greater than 800 nm, or greater than 700 nm, or greater than 600 nm, or greater than 500 nm, or greater than 400 nm, or greater than 300 nm, or greater than 200 nm, or greater than 100 nm.

In one embodiment, the first solution-second solution mixture (e.g., solvent solution-antisolvent mixture) is contained in an inlet reservoir of a high shear dispersion system, prior to introduction into the high shear zone of the device. In a further embodiment, the inlet reservoir is integrated with a temperature controller to control the temperature of the solvent solution-antisolvent mixture feed. The reservoir for the solvent solution can also utilize a temperature controller to control the temperature of solvent solution. In one embodiment, there is a heat exchanger on the outlet of the high pressure system to allow for control of the final product temperature.

Individual control of the temperatures of the feed liquids and final product can have deliberate advantages for controlling the reactions and precipitations. The heat exchange after the high shear dispersion process can be used to control temperature and avoid over heating the solution with multiple passes through the high shear dispersion system. In one embodiment, the temperature of the mixture increases 1-2° C. per pass for each 1,000 psi of process pressure. Multiple passes can be accomplished, e.g., by positioning the outlet of the high shear dispersion system in, or in close proximity to, the inlet reservoir or inlet feed. This allows for batch and continuous processes, and the ability to break down agglomerates that might form during the process. The solvent solution addition can be stopped at any time and solvent removal steps can be taken during the process.

High shear dispersion systems are known in the art, and for example and without limitation include Microfluidics Reaction Technology crystallization processor (“MRT,” Microfluidics International, Newton, Mass.), rotor-stator mills, colloid mills, homogenizers, or conventional microfluidizers. Shear rates associates with these devices are provided in Table 5 below.

Useful high shear dispersion systems herein, include systems with micro-channels that are suitable to allow the mixtures provided herein to flow through the channels without clogging. Any commercially available apparatus may be used herein, such as the Microfluidics Reaction Technology (MRT), MICROFLUIDIZER® homogenizers, commercially available from Microfluidics Corp. of Newton, Mass.; NANOMIZER™ homogenizers commercially available from Nanomizer, Inc. of Tokyo, Japan, and ULTIMIZER™ homogenizers commercially available from Sugino Machine Ltd. of Toyama, Japan; SONIFIER® homogenizers commercially available from Branson Ultrasonics Corp. of Danbury, Conn.; the DeBEE™ family of homogenizers commercially available from Bee International, Inc. of South Easton, Mass., or various others. The high shear dispersion systems may or may not use submerged jet impingement.

In one embodiment, a high shear device combines high tip speeds with a very small shear gap to produce significant friction on the material being processed. The tip speed of a high shear dispersion system of the invention, in one embodiment, is at least about 3 msec, at least about 4 msec, at least about 5 msec or at least about 6 msec.

TABLE 5

| Device | Sheer Rate (1/s) |
|---|---|
| Rotor-stator Mixer | 5,000 to 500,000 |
| Colloid Mill | 5,000 to 550,000 |
| Homogenizer | 5,000 to 650,000 |
| Microfluidizer | 5,000 to 10,000,000 |

The particles provided after the high shear dispersion step of the process of the present invention typically have a narrow particle size distribution, typically an approximately unimodal particle size distribution. The average particle size, depending on the materials used (i.e., the composition of the first and second solutions, e.g., the solvent, solute, and antisolvent) and process conditions varies from about 100 nm to about 10 μm, including about 100 nm, 200 nm, 300 nm, 400 nm, 500 nm, 600 nm, 700 nm, 800, nm, 900 nm, 1 μm, 2 μm, 3 μm, 4 μm, 5 μm, 6 μm, 7 μm, 8 μm, 9 μm, or about 10 μm, inclusive of all ranges and subranges therebetween.

The particle size distributions of the particles prepared by the method of the present invention are typically less than about one decade wide, e.g., substantially all particles are between about 10 nm and 100 nm, 20 nm and 200 nm, 30 nm and 300 nm, 100 nm and 1.0 micron, 1 micron and 10 microns, 10 microns and 100 microns, etc.

### Microfluidizer

One embodiment for achieving high shear dispersion of the first solution-second solution mixture (e.g., solvent solution-antisolvent mixture) is with the use of a microfluidizer. In one microfluidizer embodiment, the mixture is introduced into the high shear zone of a microfluidizer device via a high pressure inlet. The high shear zone, in one embodiment, may or may not use submerged jet impingement, and comprises one or more channels that having a diameter of about 1 μm to about 500 μm, or a diameter of about 10 μm to about 400 μm, or a diameter of about 20 μm to about 300 μm, or a diameter of about 30 μm to about 200 μm, or a diameter of about 40 μm to about 100 μm, or a diameter of about 50 μm to about 75 μm, or a diameter of about 50 μm to about 300 μm, or a diameter of about 100 μm to about 200 μm.

In one embodiment, the mixture proceeds through the high shear zone at a velocity of at least about 50 m/s, at least about 100 m/s, at least about 200 m/s, at least about 300 m/s at least about 400 m/s, at least about 500 m/s, or at least about 600 m/s.

One representative microfluidizer is the M-110EH-30 MICROFLUIDIZER®. Accordingly, in one embodiment, the process of the invention is conducted utilizing the M-110EH-30 MICROFLUIDIZER® processor from Microfluidics Corp., which can achieve continuous operating pressures of up to 30,000 psi (275.8 MPa). The M-110EH-30 MICROFLUIDIZER® processor directs the mixture through one or more (typically two) chambers containing precisely defined micro-channels under high pressure via an intensifier pump, separating the mixture into a plurality of mixture streams (typically two) in a “Y” interaction chamber, and causing the mixture streams to collide with each other within the interaction chamber. In one embodiment, the high pressure stream is split and recombines causing the two streams to collide with each other. The streams also collide with the walls of the channels in the interaction chamber. These collisions occur at high pressure and high velocity such that the solute particles, in one embodiment, fracture upon impact, shear, and under some circumstances cavitation occurs. In some embodiments, the fracture, shear or caviation results in solute particle size reduction. The M-110EH-30 MICROFLUIDIZER® processor is capable of running at a pressure of at least about 2,000 psi, more preferably 25,000 psi and most preferably at its maximum pressure of about 30,000 psi, resulting in high velocity collisions of the mixture streams.

### Microfluidics Reaction Technology (MRT)

MRT is based on microfluidizer processor technology that has been used for decades for particle size reduction (Panagiotuou et al. (2009). Ind. Eng. Chem. Res. 48, pp. 1761-1771). MRT added feed rate controls for multiple reactant streams, and also controls for location and mixing intensity of the streams (Id.). The reaction chamber exploits impinging jet technology to provide a micro-mixing environment in a microliter reaction volume. Shear and impact disperse the reactant streams into submicrometer eddies that have very high interfacial surface area, thereby rapidly developing homogenous conditions within the reaction chamber (Id.). This approach can also use a single stream interaction chamber for certain applications.

### Rotor-Stator Mills

A rotor-stator mill has two main components, a rapidly rotating blade (rotor) positioned with a static head or tube (stator) containing slots or holes. When introduced into the mill, because the rotor is rapidly rotating and only a narrow gap exists between the rotor and stator, the solvent solution and antisolvent are subjected to turbulence, caviation and scissor like mechanical shearing. The product, in one embodiment, is recirculated through the mill two times, three times, four times, five times, six times, seven times, eight times, nine times or ten times.

In one embodiment, the rotor speed of the mill is doubled for each halving of the rotor diameter. In choosing a rotor-stator mill, the tip velocity should be considered as well as the rpm of the motor. Other factors such as rotor-stator design, which there are many, materials used in construction, and ease of leaning are also important factors to consider in selecting a rotor-stator mill. Additionally, integration with the initial mixing step is also an important consideration when choosing a rotor-stator mill for use with the present invention.

### Colloid Mills

In one embodiment, a colloid mill is used as the high-shear dispersion device. A colloid mill applies high levels of hydraulic shear to the first solution-second solution mixture (e.g., solvent solution-antisolvent mixture). In one embodiment, the outlet stream of the colloid mill is fed back to the inlet of the colloid mill, to allow for recycling of the high-shear dispersion process. In one embodiment, the high tip speed of the colloid mill, combined with a small shear gap, produces intense friction on the material being processed. The mixed product generated by the colloid mill, in one embodiment, is recirculated through the colloid mill two times, three times, four times, five times, six times, seven times, eight times, nine times or ten times.

As described above, any high shear dispersion system, for example, one of the systems described above is amenable for use with the present invention. Additionally, the high shear dispersion can be repeated two times, three times, four times, five times, six times, seven times, eight times, nine times or ten times. This can be accomplished, for example, by introducing the outlet of the high shear dispersion system into the inlet reservoir, or by placing the outlet in close proximity to the inlet reservoir or inlet feed of the high shear dispersion system.

### Apparatuses of the Invention

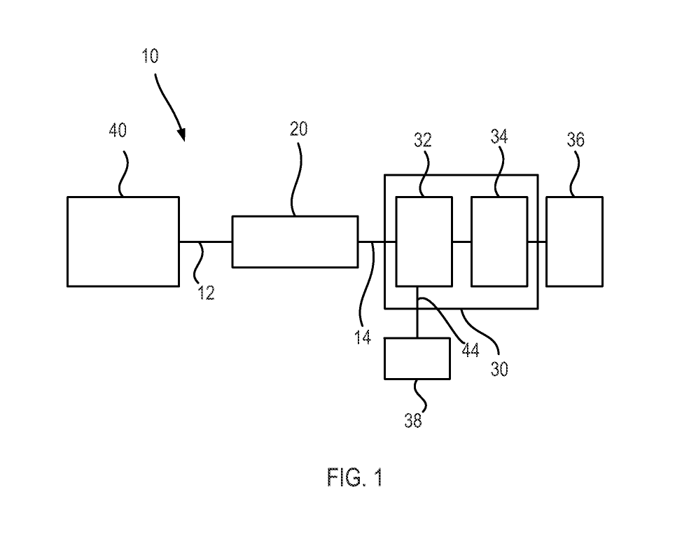

FIG. 1 is a schematic illustration of an apparatus 10 according to an embodiment of the invention. The apparatus 10 is configured to prepare a particulate material and to control the particle size and particle size distribution of the particulate material. More specifically. In some embodiments, the apparatus 10 can be configured to facilitate preparation of a crystalline or amorphous material, such as by facilitating the mixing of a solvent and antisolvent. In some embodiments, the apparatus 10 is configured to facilitate preparation of metallic particles, such as by facilitating the mixing of a solution of metal ions with a solution of a reducing agent. In some embodiments, the apparatus 10 is configured to facilitate preparation of a salt, such as by facilitating the mixing of an acid solution and a base solution (e.g., such that the salt solubility is less than the acid base solubility). In some embodiments, the apparatus 10 is configured to facilitate preparation of an emulsion, a liposome, an encapsulation, or the like. For example, the apparatus 10 can be configured to prepare a material according to a method as described herein.

The apparatus 10 includes a mixing device 20 and a high shear dispersion device 30. The mixing device 20 is fluidically coupled to a reservoir 40 by a fluid conduit 12. In some embodiments, the fluid conduit 12 is an elongate member defining a lumen configured to permit a fluid to be conveyed from the reservoir 40 to the mixing device 20. The fluid conduit 12 can be, for example, a tube, a pipe, a cannula, or another suitable mechanism for fluid conveyance. The reservoir 40 is configured to contain the fluid. More specifically, the reservoir 40 is configured to contain a solution of a solute dissolved in a solvent (also referred to herein as the “solvent solution”). The solvent solution can include any suitable solute and any suitable solvent described herein.

The mixing device 20 is configured to mix the solvent solution with an antisolvent (or the substitutions, as described above, for example, a reducing agent solution). For example, the mixing device 20 can be configured to perform the initial mixing step, as described in detail above. In some embodiments, the mixing device 20 is configured to mix and/or disperse the solvent solution such that the solvent solution is changed into the form of a fine spray, vapor, or otherwise very small drops or droplets. In this manner, the mixing device 20 can be characterized as being configured to atomize the solvent solution. The mixing device 20 is also configured to convey or inject the mixed, dispersed (or atomized) solution (e.g., in the form of the fine spray, vapor, drops or droplets) into a reservoir 32 or inlet containing an amount of antisolvent. In some embodiments, the mixing device 20 is configured to convey the solution into the antisolvent in the reservoir 32 or inlet substantially immediately following mixing or dispersion (or atomization) of the solution. In other words, as the mixing device 20 converts the solution into the spray form, the solution is sprayed or injected into the antisolvent in the reservoir 32 or inlet of the high shear device.

The mixing device 20 can include any suitable mechanism for mixing, dispersing, or atomizing the solution. For example, in some embodiments, the mixing device includes at least one of a sonicator, a venturi, a nebulizer, an atomizer, an in-line mixer, or a combination thereof. Said another way, in some embodiments, the mixing device 20 can be or include any suitable mixing device or mechanism described herein, including, for example, an ultrasonic device, a Venturi device, an in-line mixer, a spray nebulizer, or another spray device using air or gases to mix and/or disperse the solvent solution into the antisolvent.

The mixing device 20 is coupled to the high shear dispersion device 30. For example, the mixing device 20 is fluidically coupled to the second reservoir 32 of the dispersion device 30 by a fluid conduit 14. In some embodiments, the fluid conduit 14 is an outlet port of the mixing device 20. In some embodiments, the fluid conduit 14 can be similar in many respects to fluid conduit 12, described above. In some embodiments, the fluid conduit 14 can be an inlet port of the dispersion device 30. In still other embodiments, the fluid conduit 14 can be an integral component of the mixing device 20. For example, the mixing device 20 can include a spray device configured to disperse the solvent solution into the antisolvent and the spray device can include a fluid conduit 14 in the form of a spray nozzle.

The high shear dispersion device 30 includes the reservoir 32, a high-energy zone 34, and, in some embodiments, a product chamber 36. As noted above, the reservoir 32 is configured to contain the antisolvent. The antisolvent can be any suitable antisolvent, as described in detail herein. The reservoir 32 can be, for example, a feed line, a fluid chamber, an inlet conduit, or the like. In some embodiments, the apparatus 10 includes a reservoir 38 that is fluidically coupled to the reservoir 32 of the dispersion device 30 by a fluid conduit 44. As such, the antisolvent can be disposed in the reservoir 38 and selectively conveyed to the reservoir 32 of the dispersion device 30 by an operator of the apparatus 10.

The reservoir 32 of the high shear dispersion device 30 is in fluid communication with the high-energy zone 34 of the dispersion device. The dispersion device 30 is configured to convey the solution-antisolvent mixture from the reservoir 32 through the high-energy zone 34. The high-energy zone 34 is configured to facilitate dispersion of the solution-antisolvent mixture. More specifically, the high-energy zone 34 is an area within the dispersion device 30 at which the solution-antisolvent mixture is exposed to high shear forces capable of dispersing the mixture, for example, to form a suspension having a desired particle size distribution. For example, in an embodiment in which the dispersion device 30 includes a homogenizer, the high-energy zone 34 can include an area proximate to a homogenizer valve where a feed of the solution-antisolvent mixture (e.g., from the reservoir 32) has a high pressure due to a reduced orifice size and/or where the feed is moved through a flow pathway from the reduced-sized orifice through a valve and is engaged with an impact ring disposed about the valve.

In some embodiments, the dispersion device 30 is configured to convey the solvent solution-antisolvent mixture from the second reservoir 32 to the high-energy zone 34 before the mixture forms particles with an average equivalent spherical diameter of greater than 10 μm, or greater than 9 μm, or greater than 8 μm, or greater than 7 μm, or greater than 6 μm, or greater than 5 μm, or greater than 4 μm, or greater than 3 μm, or greater than 2 μm, or greater than 1 μm, or greater than 900 nm. In this manner, in one embodiment, high shear dispersion occurs when the largest particle is about 10 μm or less in diameter. In another embodiment, high shear dispersion of the solution takes place when the average particle is about 9 μm or less in diameter, about 8 μm or less in diameter, about 7 μm or less in diameter, about 6 μm or less in diameter, about 5 μm or less in diameter, about 4 μm or less in diameter, about 3 μm or less in diameter, 2 μm or less in diameter, or about 1 μm or less in diameter. By exposing the solvent solution-antisolvent mixture to the high-energy zone 34 when the largest particle is about 10 μm or less, the dispersion device 30 of the apparatus 10 is configured to form a suspension that has a substantially narrow particle size distribution.

The high shear dispersion device 30 can include at least one of a homogenizer, a rotor-stator mixer, a colloid mill, a microfluidizer, another suitable dispersion mechanism, including those mechanisms described herein, or a combination thereof. For example, the dispersion device 30 can include a homogenizer configured for at least one of ultrasonic homogenizing, pressure homogenizing, mechanical homogenizing, or a combination thereof.

The high-energy zone 34 of the high shear dispersion device is fluidically coupled to a product chamber 36. The product chamber 36 is configured to receive the dispersed solution-antisolvent mixture after it is conveyed through the high-energy zone 34 of the dispersion device 30. In some embodiments, an auxiliary chamber is disposed between the high-energy zone 34 and the product chamber 36. For example, the auxiliary chamber can be configured to facilitate modification of the process for performance or robustness.

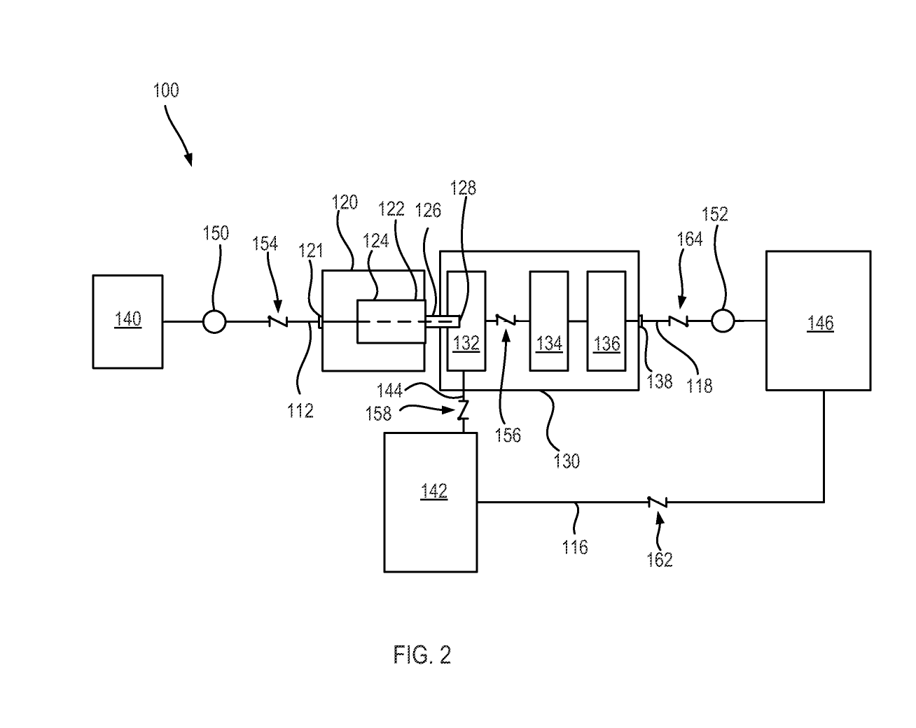

As schematically illustrated in FIG. 2, an apparatus 100 according to an embodiment is configured to prepare a particulate material (such as a crystalline or amorphous material, metal particles, or a salt), an emulsion, a liposome, an encapsulation, or the like, as described herein, and to control the particle size and particle size distribution of the particulate material. For example, the apparatus 100 can be configured to prepare a particulate material according to a method as described herein. In some embodiments, the apparatus 100 is configured to prepare the crystalline, amorphous, or two multiphase material in a continuous or batch manor. The apparatus 100 can be similar in many respects to apparatus 10 described in detail above. The apparatus 100 is configured to perform an initial mixing in which a solvent solution is mixed with an antisolvent, for example by dispersing the solvent solution into the antisolvent. The apparatus 100 is also configured for high shear dispersion of the solvent solution-antisolvent mixture such that a suspension is formed having a distribution of crystals of the solute.

The apparatus 100 includes a mixing device 120, a high shear dispersion device 130, a first reservoir 140, a second (or antisolvent) reservoir 142 and a third (or product) reservoir 146. Optional temperature control can be provided for all three reservoir system to optimize the process for specific application. The first reservoir 140 contains the solvent solution, which includes a solute dissolved in a solvent. The solvent solution can be any suitable solvent solution, including a solution of a solute and solvent described herein. For example, the solvent solution can include a pharmaceutical solute. A fluid conduit 112 is configured to convey the solvent solution from the first reservoir 140 to the mixing device 120. The fluid conduit 112 can be coupled to an inlet port 121 of the mixing device 120. In the embodiment illustrated in FIG. 2, a metering device 150 is configured to facilitate movement of the solvent solution from the first reservoir 140 to the mixing device 120. The metering device 150 can include, for example, a pump. The metering device 150 can generate a suction to draw fluid from the first reservoir 140 and/or generate a pressure to convey fluid towards the mixing device 120. An optional valve 154 is coupled to the fluid conduit 112 to control movement of fluid between the first reservoir 140 and the mixing device 120 or the pump itself can control the movement without the use of a valve. More specifically, the optional valve 154 is configured to help prevent backflow of the solvent solution from the mixing device 120 towards the first reservoir 140.

The mixing device 120, in the embodiment illustrated in FIG. 2, includes an ultrasonic device 122 configured to mix the solvent solution by sonication prior to mixing with the antisolvent. The ultrasonic device 122 can be any suitable ultrasonic device described herein. Referring to FIG. 2, the ultrasonic device 122 includes a transducer 124 and an ultrasonic horn 126. The ultrasonic device 122, and the ultrasonic horn 126 specifically, are configured to facilitate sonication of the solvent solution. Generally, the mixing device 120 is configured to convey the solvent solution from the fluid conduit 112 through a channel (represented by dashed lines in FIG. 2) defined by the transducer 124 and the ultrasonic horn 126.

In some embodiments, the mixing device 120 includes a cannula (not shown) configured to be disposed in at least a portion of the channel defined by the transducer 124 and the ultrasonic horn 126. In some embodiments, the cannula is coaxial with the channel of the ultrasonic horn 126. A first end of the cannula can be coupled to the ultrasonic transducer 124. The cannula is extended through the channel of the ultrasonic horn 126 such that a second end of the cannula is extended into a chamber (not shown) within an end 128 of the horn. The cannula can be spaced apart from vibratory elements of the ultrasonic device 122. In this manner, the cannula is not a part of the vibratory system of the ultrasonic device 122.

The mixing device 120 is configured to convey the sonicated solvent solution to an inlet reservoir 132 (e.g., a feed line, a fluid chamber, an inlet conduit, or the like) of the dispersion device 130. More specifically, as schematically illustrated in FIG. 2, the end 128 of the ultrasonic horn 126 is extended into the inlet reservoir 132 of the dispersion device 130. The inlet reservoir 132 contains an amount of antisolvent. In this manner, the ultrasonic device 122 is configured to sonicate the solvent solution and to disperse (e.g., in a high pressure stream or spray) the sonicated solvent solution from the end 128 of the ultrasonic horn 126 into the antisolvent contained in the inlet reservoir 132. As such, the ultrasonic device 122, and thus the mixing device 120, is configured to mix the solvent solution with the antisolvent.

In some embodiments, the end 128 of the horn 126 is extended into the inlet reservoir 132 such that the end 128 is suspended above the antisolvent contained in the reservoir. In some embodiments, the end 128 of the horn is extended into the inlet reservoir 132 such that the end 128 is in contact with the antisolvent contained in the reservoir.

In some embodiments, the apparatus 100 includes a trigger mechanism (not shown) configured to control conveyance of the sonicated solvent solution into the inlet reservoir 132. For example, the trigger mechanism can be configured to deliver the sonicated solution in intermittent pulses. As such, the trigger mechanism can be used to synchronize conveyance or injection of the sonicated solvent solution into the inlet reservoir 132 with conveyance of the solvent solution-antisolvent mixture from the feed line into the high-energy zone 134. In this manner, an operator can control or otherwise limit the amount of time the solvent solution-antisolvent mixture is disposed in the inlet reservoir 132 before being conveyed into the high-energy zone 134. This is advantageous to limit the particle size distribution and/or the particle size (e.g., of the solute crystals) in the dispersion, because the distribution and/or particle size may be increased relative to the time in which the solvent solution-antisolvent mixture was disposed in the inlet reservoir 132 before being dispersed.

In some embodiments, as shown in FIG. 2, the inlet reservoir 132 is in fluid communication with the second, or antisolvent, reservoir 142 of the apparatus 100 via a fluid conduit 144. The antisolvent reservoir 142 is configured to contain at least a portion of the antisolvent. The antisolvent can be conveyed via the fluid conduit 144 from the antisolvent reservoir 142 to the inlet reservoir 132 of the dispersion device 130; e.g., in preparation for preparing the particulate material. In some embodiments, the antisolvent can be injected from the antisolvent reservoir 142 into the inlet reservoir 132, such as in a high pressure flow or spray. The fluid conduit 144 can include an optional valve 158 configured to control the flow of fluid between the inlet reservoir 132 and the antisolvent reservoir 142, including, for example, the prevention of backflow of antisolvent or the solvent solution-antisolvent mixture from the inlet reservoir 132 into the antisolvent reservoir 142. As such, the antisolvent can be retained in the antisolvent reservoir 142 until it is desired by an operator of the apparatus 100 that an amount of antisolvent be disposed in the inlet reservoir 132.

The high shear dispersion device 130 also includes a high-energy zone 134 and a product chamber 136. The dispersion device 130 is configured to convey the solvent solution-antisolvent mixture from the inlet reservoir 132 to the high-energy zone 134. In this manner, the dispersion device 130 can be characterized as including a feed line configured to convey the mixture to the high-energy zone 134. In some embodiments, as illustrated in FIG. 2, the dispersion device 130 includes a valve 156 configured to control the flow of the solvent solution-antisolvent mixture between the inlet reservoir 132 and the high-energy zone 134.

The high-energy zone 134 is configured to facilitate dispersion of the solution-antisolvent mixture. More specifically, the high-energy zone 134 is an area within the dispersion device 130 at which the solvent solution-antisolvent mixture is exposed to high shear forces capable of dispersing the mixture, for example, to form a suspension having a distribution of particles (e.g., solute crystals). The high-energy zone 134 will be understood by those skilled in the art based upon the selected dispersion device. For example, the dispersion device can be or include any suitable dispersion mechanism, including but not limited to that found in the dispersion devices described herein, such as a homogenizer, a colloid mill, a rotor-stator, or a microfluidizer.

The dispersion device 130 is configured to convey the dispersed solvent solution-antisolvent mixture from the high-energy zone 134 into the product chamber 136. The product chamber 136 can be configured to modify the energy generated during dispersion of the solvent solution-antisolvent mixture through the high-energy zone 134. The product chamber 136 of the dispersion device 130 is fluidically coupled to a product reservoir 146 via a fluid conduit 118. In the embodiment illustrated in FIG. 2, the fluid conduit 118 is coupled to the dispersion device 130, and the product chamber 136 specifically, by an outlet port 138. A metering device 152, e.g., a pump, is configured to control conveyance of fluid (i.e., the dispersed solvent solution-antisolvent mixture) from the dispersion device 130 to the product reservoir 146. The metering device 152 can be similar in many respects to the metering device 150 described above. The dispersed solvent solution-antisolvent mixture can be conveyed from the dispersion device 130 through the fluid conduit 118 into the product reservoir 146; e.g., for storage of the suspension of particles formed by the dispersed mixture.

The product reservoir 146 is fluidically coupled to the antisolvent reservoir 142 via a fluid conduit 116. In this manner, at least a portion of the dispersed solution-antisolvent mixture can be circulated from the product reservoir 146 to the antisolvent reservoir 142, from which it can then be conveyed to the inlet reservoir 132. As such, a solution-antisolvent mixture can be selectively processed multiple times through the high-energy zone 134 of the dispersion device. In some embodiments, the solution-antisolvent mixture can be selectively passed or recirculated through the high-energy zone 134 one, two, three, four, or more times. The fluid conduit 116 can include a valve 162 configured to control the flow of fluid between the product reservoir 146 and the antisolvent reservoir 142. More specifically, recirculation of the dispersed solvent solution-antisolvent mixture can be controlled by the valve 162 in the fluid conduit 116. Furthermore, the valve 162 can help prevent flow of fluid from the antisolvent reservoir 142 to the product reservoir 146 via the fluid conduit 116.

While various embodiments of the invention have been described above, it should be understood that they have been presented by way of example only, and not limitation. Where methods described above indicate certain events occurring in certain order, the ordering of certain events may be modified, unless clearly indicated otherwise. Additionally, certain of the events may be performed concurrently in a parallel process when possible, as well as performed sequentially as described above. Thus, the breadth and scope of the invention should not be limited by any of the above described embodiments. While the invention has been particularly shown and described with reference to specific embodiments thereof, it will be understood that various changes in form and details may be made.

For example, although the apparatus 100 is illustrated and described herein as including metering devices 150, 152, in other embodiments, an apparatus for preparing a particulate material can include a flow metering system having a different configuration. For example, in some embodiments, an apparatus for preparing a particulate material can include a metering device disposed at a different position or portion of the apparatus. For example, in some embodiments, the metering device 152 can be disposed between the valve 156 and the high-energy zone 134 of the dispersion device 130. In another example, the apparatus 100 can include no metering device between the dispersion device 130 and the product reservoir 146. In yet another example, a metering device can be disposed between the mixing device and the dispersion device to control the flow of fluid (i.e., the sonicated solution) therethrough. In another example, a metering device can be disposed between the antisolvent reservoir 142 and the inlet reservoir 132. In still another example, a metering device can be disposed between the product reservoir 146 and the antisolvent reservoir 142. Additionally, in some embodiments, an apparatus for preparing a particulate material can any suitable number of metering devices, such as one, three, four, or more metering devices.

Although the metering devices 150, 152 are illustrated and described herein as being inline with the fluid conduits 112, 118 at a certain position, in other embodiments, an apparatus for preparing a particulate material can include a metering device fluidically coupled in a different manner and/or position. For example, the metering device 150 can be fluidically coupled to fluid conduit 112 between the valve 154 and the mixing device 120. Moreover, the metering devices can include any suitable mechanism for controlling fluid flow, including, for example, any suitable type of pump. For example, in some embodiments, an apparatus can include a piston pump, an inline pump, or the like.

In another example, although the dispersion device 130 is illustrated and described herein as including a fluid conduit 116 for recirculation of the dispersed mixture from the product reservoir 146 to the antisolvent reservoir 142, in other embodiments, a dispersion device can be differently configured for recirculation of the dispersed mixture for multiple processing. For example, in some embodiments, the dispersion device includes a heat exchanging coil through which at least a portion of the dispersed mixture is conveyed. An outlet of the heat exchanging coil can be fluidically coupled to at least one of the antisolvent reservoir 142 or the inlet reservoir 132 such that the portion of the dispersed mixture can be recirculated into the antisolvent reservoir or the inlet reservoir for additional passes through the high-energy zone.

Although the inlet reservoir 132, the high-energy zone 134, and the product chamber 136 are schematically illustrated as being discrete elements, in some embodiments, a dispersion device can include the inlet reservoir, high-energy zone, and product chamber in any suitable configuration. Additionally, although the high shear dispersion device 130 is illustrated and described as including product chamber 136, in other embodiments, a high shear dispersion device is configured to without such a product chamber. For example, in some embodiments, a high shear dispersion device is configured to convey a fluid from the high-energy zone (e.g., similar to high-energy zone 134) into a product reservoir (e.g., similar to product reservoir 146).

In another example, although the apparatus 100 is illustrated and described as including a valve (e.g., valve 154, 156, 158, 162, 164) being disposed at a certain position within the apparatus, in other embodiments, an apparatus for preparing a particulate material can include a valve disposed at a variety of positions. Although a valve (e.g., valve 154, 156, 158, 162, 164) described herein are illustrated in FIG. 2 as being a check valve, in other embodiments, any suitable type of valve may be used. Furthermore, although apparatus 100 is illustrated an described as including five valves, in other embodiments, apparatus 100 can include any suitable number of valves. For example, in some embodiments, an apparatus can include one, two, three, four, six, or more valves, or none. As an example, although apparatus 100 includes valve the 156 between the inlet reservoir 132 and the high-energy zone 134, in other embodiments, an apparatus may include no valve between the inlet reservoir and the high-energy zone.

Although the apparatus 100 is illustrated and described herein as including a homogenizer 122 in the mixing device 120, in other embodiments, an apparatus can have a mixing device with a another suitable mixing mechanism. In some embodiments, for example, an apparatus for preparing a particulate material can include a mixing device 220 that is or includes a Venturi device.

### EXAMPLES

The present invention is further illustrated by reference to the following Examples. However, it should be noted that these Examples, like the embodiments described above, are illustrative and are not to be construed as restricting the enabled scope of the invention in any way.

### Example 1

### Crystallization of Naproxen

A submicron dispersion of naproxen was prepared using the enhanced initial dispersion followed by a secondary high shear mixing process. The following equipment was used for the experiment:

(1) An Ivek Digispense 3009 controller with an AP motor/base assembly and a AA pump body.

(2) A Micronoson Ultrasonic Cell Distruptor, XL 100 watt ultrasonic controller and a Misonix 22.5 kHz ultrasonic transducer horn with a coaxillal center feed.

(3) Microfluidizer—Microfluidics M-110EH-25 high shear processor with a 75 micron F-20Y interaction chamber.

(4) Horiba LA-950 Laser Diffraction Particle Size Analyzer.

The Ivek Digispense pump speed was calibrated to 20 mL/min.

The end of the ultrasonic horn, also referred to as an ultrasonic probe, was suspended in air, the power to the Ivek pump and the ultrasonic probe was turned on. Ultrasonic power was set to 20 watts. The height of the ultrasonic probe dispense tubing was adjusted until the feed liquid was dispersed as small droplets, thereby creating a fine spray. The tubing was fixed at this height, as above and below this height, dispensed liquid simply drips from the probe.

The tip of the ultrasonic transducer horn was secured just above the check valve in the feed reservoir. The antisolvent was a 0.5% Triton X-100 in dionized water, and 190 mL of the antisolvent was added to the inlet reservoir of the Microfluidizer. Naproxen has a tendency to agglomerate, therefore, nonionic Triton X-100 surfactant was used as the antisolvent.

DMSO was used as the solvent and it contained 13.75 g naproxen per 100 mL solvent. An ice bath was used to chill the sample to avoid over heating with multiple passes.

The microfluidizer pressure was set to 24,000 psi, using the antisolvent. This resulted in a flow rate of 380 mL/min. The outlet from the cooling coil of the microfluidizer was placed in the inlet reservoir to allow for recycling of the process. The Ivek Pump was set for 20 mL/min.

With the Microfluidizer running at 24,000 psi in a recirculation mode the Pump and Ulrasonic Probe were simutaneously turned on for 30 seconds and then immediately turned off. A single pass sample was collected for particle size analyzsis. This sample contained 1.375 grams of naproxen in the mixed solution of DMSO & 0.5% triton X-100 DI water. The Microfluidizer continued to run for an additional 150 seconds for a total of 180 seconds. After six passes, a sample was collected to evaulate particle size.

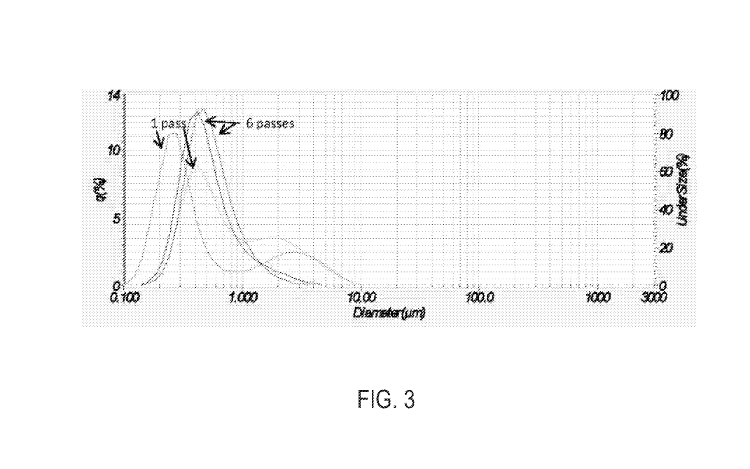

The results of this experiment are provided in FIG. 3 and Table 6. The graph presented in FIG. 3 is a plot of naproxen particle diameter (x axis) vs. frequency of occurrence of the particle diameter (y axis). Table 6 provides the mean and median particle size for each run. Regardless of how many passes were employed, the median naproxen particle size was less than 1 μm in diameter. Additional passes increased the reproducibility of the method, as shown by the overlapping curves.

TABLE 6

| | Mean Particle Size | Median particle size |
|---|---|---|
| Passes | (μm) | (μm) |
| 1 | 1.02077 | 0.3267 |
| 1 | 1.25661 | 0.64592 |
| 6 | 0.60509 | 0.50673 |
| 6 | 0.60600 | 0.46025 |

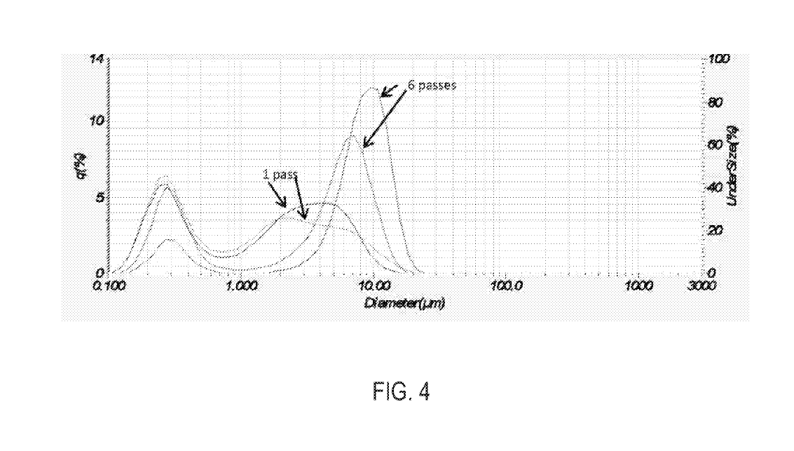

FIG. 4 and Table 7 show the result from an identical experiment, as set forth above, except that no ultrasonic step was employed. The results demonstrate that ultrasonication leads to more reproducible particle size distributions, a narrower particle size distribution, and smaller particle sizes.

TABLE 7

| | Mean Particle Size | Median particle size |
|---|---|---|
| Passes | (μm) | (μm) |
| 1 | 4.64975 | 4.78376 |
| 1 | 8.19928 | 8.32399 |
| 6 | 2.4845 | 1.21359 |
| 6 | 2.46975 | 1.69571 |

The monomodal distribution after 6 passes has a larger median than the single pass. Without wishing to be bound by theory, this may indicate that the finer mode (i.e., the first peak in FIG. 4, corresponding to the first pass experiment) was agglomerating or growing while the coarse mode (i.e., the second peak in FIG. 4, corresponding to the first pass experiment) was broken down by the additional processing.

Patents, patent applications, publications, product descriptions, and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

What is claimed is:

1. A method of continuously preparing a crystalline or amorphous material comprising:
mixing a solvent solution comprising a solute dissolved in a solvent, with a miscible non-solvent system, thereby providing a solvent solution-antisolvent mixture; and
introducing the mixture into the high-energy zone of a high shear dispersion system and dispersing the solvent solution-antisolvent mixture prior to significant crystal growth, whereby the dispersed mixture forms a suspension of crystals of the solute; and wherein significant crystal growth has occurred when the average equivalent spherical diameter of at least one crystal in the solution is greater than 10 μm.

2. The method of claim 1, wherein said mixing step comprises processing the solvent solution with a sonicating device or an air nozzle, and dispersing the processed solvent solution into the antisolvent.

3. The method of claim 2, wherein said mixing step further comprises directly feeding the sonicated solvent solution into the inlet reservoir of the high shear dispersion system.

4. The method of claim 1, wherein the high speed dispersion step is carried out in a microfluidizer, homogenizer, colloid mill or rotor-stator mixer.

5. The method of claim 1, wherein the mixing step comprises in-line mixing of the solvent solution, atomization of the solvent solution, or mixing the solvent solution in a Venturi device.

6. The method of claim 5, wherein the time between the mixing and introducing steps is selected from 10 seconds, 9 seconds, 8 seconds, 7 seconds, 6 seconds, 5 seconds, 4 seconds, 3 seconds, 2 seconds, 1 seconds, 0.5 seconds, 0.1 seconds and 0.01 seconds.

7. The method of claim 1, wherein the solvent system comprises one or more solvents selected from the group consisting of water, ethanol, isopropanol, DMSO, and combinations thereof.

8. The method of claim 1, wherein the solute is an active pharmaceutical ingredient.

9. An apparatus for preparing a particulate material, comprising:
a mixing device fluidically coupled to a first reservoir containing a first solution, the mixing device configured to convey the first solution into a second reservoir containing a second solution such that the first solution and the second solution are mixed; and
a high shear dispersion device fluidically coupled to the mixing device, the high shear dispersion device configured to convey the first solution-second solution mixture from the second reservoir through a high-energy zone of the high shear dispersion device, the high-energy zone configured to facilitate dispersion of the first solution-second solution mixture.

10. The apparatus of claim 9, wherein the mixing device is configured to atomize the first solution, the mixing device configured to inject the atomized first solution into the second solution in the second reservoir.

11. The apparatus of claim 9, wherein the mixing device is configured to convey the first solution through a channel defined by an ultrasonic horn of the mixing device, the ultrasonic horn configured to sonicate the first solution.

12. The apparatus of claim 9, wherein at least a portion of the mixing device is extended into the second reservoir.

13. The apparatus of claim 9, wherein the mixing device includes at least one of a sonicator, a venturi, a nebulizer, an atomizer, or an in-line mixer.

14. The apparatus of claim 9, wherein the high shear dispersion device includes at least one of a homogenizer, a rotor-stator mixer, a colloid mill, or a microfluidizer.

15. A suspension of nanoparticles prepared by the method of claim 1, wherein the particle size distribution less than one decade wide.

16. The suspension of nanoparticles of claim 15, wherein the average particle size is 1 μm.

17. The suspension of nanoparticles of claim 16, wherein the nanoparticles comprise an active pharmaceutical ingredient.

18. A method of continuously preparing a particulate material comprising:
mixing a first solution comprising a solute dissolved in a solvent, with a second solution, thereby providing a first solution-second solution mixture; and
introducing the mixture into the high-energy zone of a high shear dispersion system and dispersing the first solution-second solution mixture prior to significant particulate growth, whereby the dispersed mixture forms a suspension of particles of the solute; and wherein significant particulate growth has occurred when the average equivalent spherical diameter of at least one particle in the solution is greater than 10 μm.

19. The method of claim 18, wherein the first solution is a solvent solution and the second solution is a miscible antisolvent solution.

20. The method of claim 18, wherein the first solution is a solvent solution and the second solution is an immiscible antisolvent solution.

21. The method of claim 18, wherein the first solution is a solvent solution comprising an inorganic salt as the solute, and the second solution is a reducing agent solution.

22. The method of claim 18, wherein the first solution is an acidic solution and the second solution is a basic solution.
